(19) United States
(12) Patent Application Publication
(10) Pub. No.: US 2007/0161019 A1
(43) Pub. Date: Jul. 12, 2007

(54) Method for detecting cancer and a method for suppressing cancer

(76) Inventors: Johji Inazawa, Tokyo (JP); Issei Imoto, Tokyo (JP); Jun Inoue, Tokyo (JP); Akiko Furihata, Tokyo (JP); Sana Yokoi, Tokyo (JP); Hideaki Tanami, Tokyo (JP); Hiroyuki Izumi, Tokyo (JP); Kuniyasu Saigusa, Tokyo (JP); Shin Hayashi, Tokyo (JP); Hisashi Takada, Tokyo (JP); Ayako Suzuki, Tokyo (JP); Itaru Sonoda, Tokyo (JP)

Correspondence Address: BIRCH STEWART KOLASCH & BIRCH, PO BOX 747, FALLS CHURCH, VA, 22040-0747, US

(21) Appl. No.: 11/591,637

(22) Filed: Nov. 2, 2006

(30) Foreign Application Priority Data
Nov. 4, 2005 (JP) 2005-304497

(51) Int. Cl. C12Q 1/68; A61K 48/00

(52) U.S. Cl. 435/6; 514/44

(57) ABSTRACT

An object of the invention is to find a cancer-associated gene to be used as an index for detecting canceration of cells and degree of malignancy of cancer, so as to to provide a method for detecting cancer using the cancer-associated gene as an index and provide a method of suppressing/treating cancer using the cancer-associated gene as essential part. According to the present invention, specific genes which are amplified or deleted in lung cancer as compared with normal cell have been collectively found, and a method for detecting cancer using amplification or deletion of these cancer-associated genes as an index is provided. Further, cancer can be suppressed by introducing a gene which is deleted in cancer cells amond these cancer-associated genes into cancer and inhibiting the transcription product of the gene amplified.

### TECHNICAL FIELD

The present invention relates to a method of detecting canceration and malignancy of cancer using a specific cancer-associated gene as an index, and also relates to a method of suppressing/treating cancer using a specific cancer-associated gene as essential part.

### Background Art

A mortality rate of cancer is presently the top end in Japan and occupies one third of the total mortality causes. The mortality rate of cancer goes on increasing and is predicted to occupy about 50% in 10 years. It has been elucidated that cancer is caused and aggravated due to accumulation of abnormalities of many genes. It has been reported that acceleration of oncogene expression and deceleration of cancer suppressor gene expression due to deletion are involved in canceration. Furthermore, it is also known that abnormalities of a gene directly involved in cell differentiation and proliferation and a gene involved in a DNA repair system are involved in canceration.

However, studies that have been hitherto conducted are not sufficient to explain the canceration mechanism in cancer patients. A group of genes involved in canceration varies depending upon the type of cancer. Furthermore, since the individual characters of cancers differ even if they belong to the same type, it has been difficult to systematically analyze the abnormality of which gene group causes cancer. Therefore, it cannot be said that a sufficient diagnostic method for the initial state of cancer and a sufficient diagnostic means for checking degree of malignancy of cancer based on genomic analysis of cancer cells have been provided.

### DISCLOSURE OF THE INVENTION

An object of the invention is to find a cancer-associated gene to be used as an index for detecting canceration of cells and degree of malignancy of cancer and to provide a method for detecting cancer using the cancer-associated gene as an index. Another object of the present invention is to provide a method of suppressing/treating cancer using the cancer-associated gene as essential part.

Generally, when a chromosomal abnormality takes place, the cell causes apoptosis to death. Therefore, proliferation of an abnormal cell does not occur in mechanism. However, in some cases, a cell having a chromosomal abnormality may happen to initiate proliferation for an unknown reason through a loophole of the biological control mechanism that should be strictly controlled, thus initiating canceration. Therefore, amplification and deletion of a genome at a chromosomal level are critical causes of canceration. In the case of amplification, expression of a gene present in the amplified genomic region is accelerated, whereas, in the case of deletion, the expression level of a gene present in the deleted genomic region is significantly decelerated. When such abnormalities are accumulated, a cell may probably cause unregulated proliferation.

Comparative genomic hybridization (CGH) is a simple and quick method, that is, the best method, for analyzing gene abnormalities associated with genomic amplification and deletion of a plurality of genes. To analyze abnormality of a gene on the genome involved in canceration and malignant alteration of cancer, it is extremely important to select a group of genes to be printed on a CGH microarray.

The present inventors screened a group of highly potential genes that may be involved in canceration from the databases “National Cancer for Biotechnology” and “University of California Santa Cruz Biotechnology.” They further subjected the DNA thus screened to BLAST search to select genes that conceivably play an important role in the onset of cancer. BAC/PAC clones containing these candidate cancer-associated genes are carefully selected and individually amplified (inexhaustibly amplified). Then, about 800 types of clones thus amplified were loaded on a substrate to form a “MCG cancer array” substrate (hereinafter also referred to “MCG cancer array”). The present invention encompasses the MCG cancer array within its technical range.

The present inventors found cancer-associated genes to be used as cancer detection indexes in several types of cancer by use of the MCG cancer array. Based on the finding, they accomplished one of the present inventions.

More specifically, the present invention provides a method of detecting (hereinafter referred to also as “the detection method of the invention”) cancer using a specific cancer-associated gene as an index. Also in the present invention, there is provided a means for suppressing/treating cancer using the cancer-associated gene. More specifically, the present invention provides a means for suppressing/treating cancer by introducing a specific deletion cancer-associated gene into a cancer cell and a means for suppressing/treating cancer by inhibiting the function of the transcriptional product (mRNA) of a specific amplification cancer-associated gene. These means for suppressing/treating cancer will be explained later.

The present invention provides a method for detecting cancer selected from lung squamous cell carcinoma, lung adenocarcinoma or lung large cell carcinoma, wherein canceration of a specimen is detected based on an index of not less than 1.5 fold amplification of at least one gene selected from the group consisting of MUC1 gene, PRCC gene, EIF4G gene, THPO gene, TERT gene, DAB2 gene, EGFR gene, ELN gene, MUC3 gene, MYC gene, PVT1 gene, FACA gene, PTPN1 gene, Livin gene, MYCN gene, ELK3 gene, BCL2L2 gene, HNF3A gene, ERBB2 gene, and CGI-147 gene; in comparison with a normal cell.

The present invention further provides a method for detecting cancer as mentioned above, wherein canceration of a specimen is detected based on an index of not less than 4 fold amplification of at least one gene selected from the group consisting of MYCN gene, MYC gene, PVT1 gene, ELK3 gene, BCL2L2 gene, HNF3A gene, ERBB2 gene, and CGI-147 gene; in comparison with a normal cell.

The present invention further provides a method for detecting non-small cell lung cancer, wherein canceration of a specimen is detected based on an index of not less than 1.5 fold amplification of at least one gene selected from the group consisting of ESP15 gene, MUC1 gene, ARHGEF2 gene, PRCC gene, ABCC5 gene, EIF4G1 gene, CDH12 gene, PC4 gene, SKP2 gene, DAB2 gene, ZNF131 gene, RAD52 gene, WNT3 gene, CDC27 gene, ABCC3 gene, PPM1D gene, ITGB4 gene, BIRC5 gene, SHGC-103396 gene, BCL2L1 gene, RBL1 gene, SRC gene, TGIF2 gene, MYBL2 gene, BIRC7 gene, ARAF1 gene, CUL4B gene, CTAG1 gene, TRIM33 gene, MYCL1 gene, RLF gene, MYC gene, CCNE1 gene, and PCTK1 gene; in comparison with a normal cell.

The present invention further provides a method for detecting cancer as mentioned above, wherein canceration of a specimen is detected based on an index of not less than 4 fold amplification of at least one gene selected from the group consisting of TRIM33 gene, MYCL1 gene, RLF gene, MYC gene, CCNE1 gene, and PCTK1 gene; in comparison with a normal cell.

The present invention further provides a method for detecting cancer selected from lung squamous cell carcinoma, lung adenocarcinoma or lung large cell carcinoma, wherein canceration of a specimen is detected based on an index of a heterozygous deletion of at least one gene selected from the group consisting of BAIAP1 gene, LZTS1 gene, AAC1 gene, BLK gene, MTAP gene, CDKN2A (p16) gene, GPC5 gene, SNRPN gene, SAMD4 gene, DCC gene, ADRBK2 gene, DBCCR1 gene, and RIZ gene, or an index of a homozygous deletion of at least one gene selected from the group consisting of CDKN2A (p16) gene, MTAP gene, DBCCR1 gene, and RIZ gene.

The present invention further provides a method for detecting non-small cell lung cancer, wherein canceration of a specimen is detected based on an index of a heterozygous deletion of at least one gene selected from the group consisting of CCK gene, UBE2E1 gene, THRB gene, RARB gene, TGFβR2 gene, MLH1gene, CTNNB1 gene, VIPR1 gene, ZNF35 gene, TGM4 gene, TDGF1 gene, PTPRG gene, FHIT gene, BAIAP1 gene, MITF gene, LOC151987 gene, EIF4E gene, NFκB gene, CCNA gene, PGRMC2 gene, VEGFC gene, MSH3 gene, RASA1 gene, TPT1 gene, RB1 gene, AATF gene, ADRBK2 gene, and NB2F1 gene, or an index of a homozygous deletion of NR2F1 gene.

The present invention further provides a method of detecting cancer wherein a non-small cell lung cancer is detected by detecting suppression of DBCCR1 gene expression in a specimen.

Preferably in the above, the detection is performed by a CGH method, DNA chip method, quantitative PCR method or real time RT-PCR method.

Preferably in the above, detection is performed by a CGH method or DNA chip method and a plurality of types of DNA fragments to be fixed onto the detection substrate are genomic DNA, cDNA or synthetic oligonucleotides.

Preferably in the above, the detection is performed by a CGH method, and a plurality of types of DNA fragments to be fixed onto the detection substrate are genomic DNA, and the genomic DNA is a gene amplification product of BAC DNA, YAC DNA or PAC DNA.

The present invention further provides a method for suppressing a non-small cell lung cancer cell, which comprises introducing a gene, whose deletion is involved in canceration of a non-small cell lung cancer cell, into a non-small cell lung cancer cell.

The present invention further provides a method for suppressing a non-small cell lung cancer cell, which comprises introducing at least one gene selected from a group consisting of MTAP gene, CDKN2A(p16) gene, DBCCR1 gene and 1RIZgene into a non-small cell lung cancer cell.

The present invention further provides a method for suppressing a small cell lung cancer cell, which comprises introducing a gene, whose deletion is involved in canceration of a small cell lung cancer cell, into a small cell lung cancer cell.

The present invention further provides a method for suppressing a small cell lung cancer cell, which comprises introducing NR2F1 gene into a small cell lung cancer cell.

The present invention further provides a method of suppressing a small cell lung cancer cell, which comprises applying, to a small cell lung cancer cell, a nucleic acid antagonizing a transcriptional product of a gene whose amplification is involved in canceration of the small cell lung cancer cell.

The present invention further provides a method of suppressing a small cell lung cancer, which comprises applying, to a small cell lung cancer, a nucleic acid antagonizing a transcriptional product of at least one gene selected from the group consisting of MYCN gene, MYC gene, PVT1 gene, ELK3 gene, BCL2L2 gene, HNF3A gene, ERBB2 gene and CGI-147 gene.

The present invention further provides a method of suppressing a non-small cell lung cancer cell, which comprises applying, to a non-small cell lung cancer cell, a nucleic acid antagonizing a transcriptional product of a gene whose amplification is involved in canceration of the non-small cell lung cancer cell.

The present invention further provides a method of suppressing a non-small cell lung cancer, which comprises applying, to a non-small cell lung cancer, a nucleic acid antagonizing a transcriptional product of at least one gene selected from the group consisting of TRIM33 gene, MYCL1 gene, RLF gene, MYC gene, CCNE1 gene and PCTK1 gene.

Preferably in the above, the nucleic acid antagonizing a transcriptional product of a gene is small interference RNA against a transcriptional poroduct mRNA, or an antisense oligonucleotide of the mRNA.

### BRIEF DESCRIPTION OF THE DRAWINGS

FIG. 1 shows an illustration of genome analysis for a normal diploid cell by use of the MCG cancer array.

FIG. 2 shows a graph showing the results of the genome analysis for a cancer cell by use of the MCG cancer array.

FIG. 3 shows an illustration showing the amplification of SKP2 gene, PC4 gene and CDH6 gene present in 5p13 of a non-small cell lung cancer cell, and the expression level of the SKP2 gene and PC4 gene.

FIG. 4 shows diagrams showing inhibition of proliferation of a small-cell lung cancer cell with addition of an SKP2 antisense oligonucleotide,

FIG. 5 shows the results of apoptosis induction of ACC-LC172 cells with addition of an SKP2 antisense oligonucleotide.

### BEST MODE FOR CARRYING OUT THE INVENTION

A. The Detection Method of the Invention

The detection according to the invention may be carried out by CGH method, DNA chip method, quantitative PCR method, or real time RT-PCR method. To detect amplification or deletion of a gene, the DNA chip method or CGH method is preferably used and the CGH method is particularly preferable. When the expression of a cancer suppressor gene (corresponding to the “deletion gene” mentioned above) is suppressed by another cause except for gene deletion, such as acceleration of methylation of a CpG island of the gene and deceleration of acetylation of a protein associated with the gene, it is preferable to employ a detection means for detecting an transcriptional product of the gene, such as the real time RT-PCR method and the DNA chip method, capable of quantifying the transcribed product of the gene.

The specimen to be subjected to the detection method of the present invention is derived from a subject and corresponds to the type of cancer to be detected. To explain more specifically, a lung biopsy specimen is used when a subject is checked for lung carcinoma.

As a preferable embodiment of the detection method of the present invention, mention may be made of application of a CGH method to a substrate on which a plurality of types of gene amplification products having a specific genome DNA region obtained from a BAC (bacterial artificial chromosome) DNA, YAC (yeast artificial chromosome) DNA, or PAC (phage artificial chromosome) DNA are individually and separately fixed. In this embodiment, amplification and deletion gene of a genomic DNA can be analyzed by the CGH method.

The amount of the BAC DNA generally obtained is too little to fix onto numerous substrates practically used as genomic DNA fixed substrates. Therefore, the DNA must be obtained as an amplified product of a gene (the amplification process of the gene is also called as “inexhaustible process”). In the inexhaustible process, BAC DNA etc., was digested with a 4-nucleotide recognition enzyme, such as RsaI, DpnI, or HaeIII, and then, an adapter was added to ligate the digested fragments. The adapter is an oligonucleotide formed of 10 to 30 nucleotdes and preferably 15 to 25 nucleotides. The double stranded chain has a complementary sequence. After annealing, the 3′ end of the oligonucleotide forming a smooth end must be phosphorylated. Then, using a primer having the same sequence as one of the oligonucleotides serving as the adaptor, amplification is performed by PCR (Polymerase Chain Reaction). In this manner, the inexhaustible process can be carried out. On the other hand, an aminated oligonucleotide having 50 to 70 nucleotides characteristic in each of the BAC DNA and the like may be used as a detection probe.

The inexhaustibly amplified BAC DNA or the like (the same in the embodiment genomic DNA, cDNA or synthetic oligonucleotide is used) is fixed onto a substrate, preferably a solid substrate, to manufacture a desired DNA fixed substrate.

Examples of the solid substrate include glass, plastic, membrane and a three-dimensional array. Preferably a glass substrate such as a slide glass is preferable. The solid substrate formed of such as glass is preferably coated by depositing poly-L-lysine, amino silane, gold, and aluminium thereon and applied by an amino group modified DNA immobilization surface treatment.

The concentration of the inexhaustibly amplified DNA mentioned above (the same in the embodiment genomic DNA, cDNA or synthetic oligonucleotide is used) to be spotted on the substrate is preferably 10 pg/μl to 5 μg/μl, and more preferably, 1 ng/μl to 200 ng/μl. The amount of the spot is preferably 1 nl to 1 μl, and more preferably, 10 nl to 100 nl. The size and shape of individual spots to be fixed on the substrate are not particularly limited; however, for example, may be a diameter of 0.002 to 0.5 mm and a circular to elliptic shape as viewed from the top. The thickness of dry spots is not particularly limited; however, may be 1 to 100 μm. The number of spots are not particularly limited; however, preferably 10 to 50,000, and more preferably 100 to 5,000. Each DNA may be spotted in the range of a singular spot to quadruplicated spots, and preferably duplicated or triplicated spots.

The dry spots may be prepared by spotting a plurality of spots of BAC DNA and the like (the same in the embodiment genomic DNA, cDNA or synthetic oligonucleotide is used) inexhaustibly amplified on a substrate by means of a spotter, and drying the spots. As the spotter, use may be made of an inkjet printer, pin array printer, and bubble-jet (registered trade mark) printer; however, an inkjet printer may be preferably used. More specifically, use may be made of GENESHOT (NGK insulators Ltd., Nagoya) and high-throughput inkjet delivery system SQ series (manufactured by Cartesian Technologies, USA), etc.

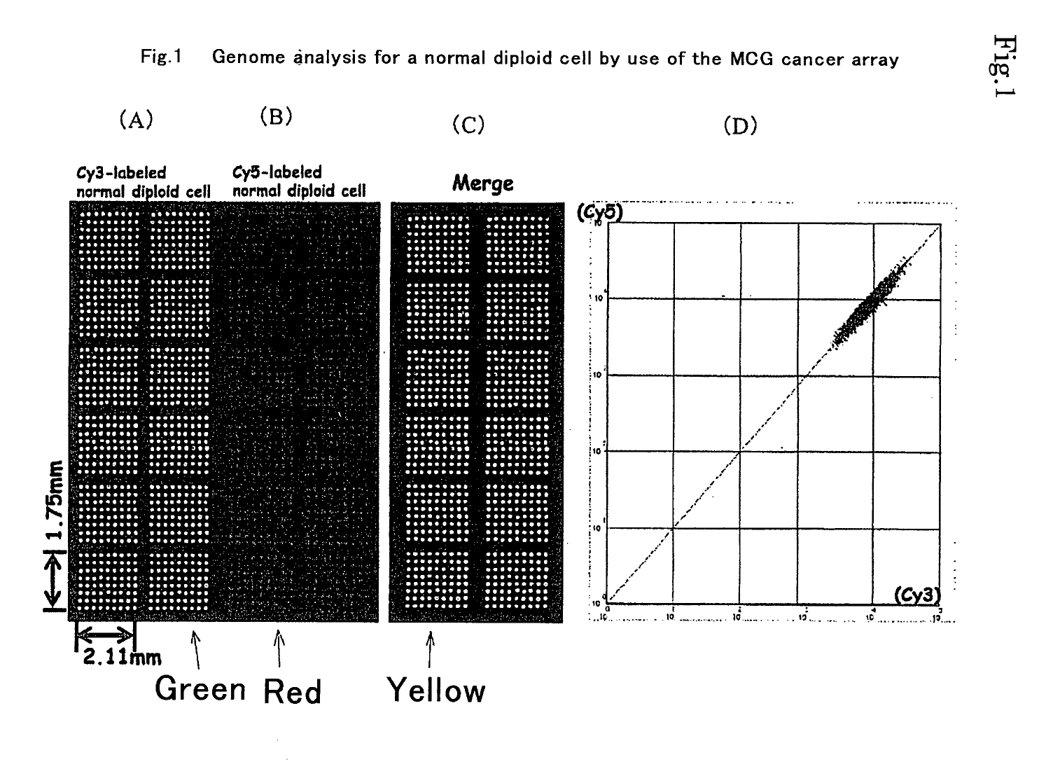

In the manner mentioned above, a desired DNA fixation substrate can be manufactured by fixing BAC DNA and the like (the same in the embodiment genomic DNA, cDNA or synthetic oligonucleotide is used) inexhaustibly amplified on a substrate, and preferably a solid substrate. Hybridization was actually performed using Cy-3 labeled genomic DNA derived from a normal diploid cell, and Cy-5 labeled genomic DNA derived from the same normal diploid cell separately on the MCG cancer array. The results are shown in FIG. 1, together with the hybridization results performed with the mixture of them (indicated by “Merge”). When Cy-3 labeled genomic DNA is used, green fluorescence is detected. When Cy-5 labeled genomic DNA is used, red fluorescence is detected. When both are mixed, yellow fluorescence is detected.

In the MCG cancer array shown in FIG. 1, 432 types of BAC DNA were printed. The BAC DNA collectively contains a group of cancer-associated genes such as oncogenes and cancer suppressor genes. In the one district of the array having 1.75 mm length and 2.11 wide, 72 DNA spots are printed. In total, 432 spots are arranged in a linear row and printed in duplicate. FIG. 1A shows the hybridization results of Cy-3 labeled normal diploid cell genomic DNA and thus all spots are green. FIG. 1B shows the hybridization results of Cy-5 labeled normal diploid cell genomic DNA and thus all spots are red. FIG. 1C (indicated “Merge” on the slide substrate) shows the hybridization results of a mixture of the Cy-3 labeled DNA and the Cy-5 labeled DNA and all spots are yellow. When the fluorescence intensity of Cy-3 is plotted on the transverse axis and that of Cy-5 is plotted on the vertical axis, all plots of signals draw a straight line and converged into an intensity of 5×10³to 5×10⁴ (FIG. 1D).

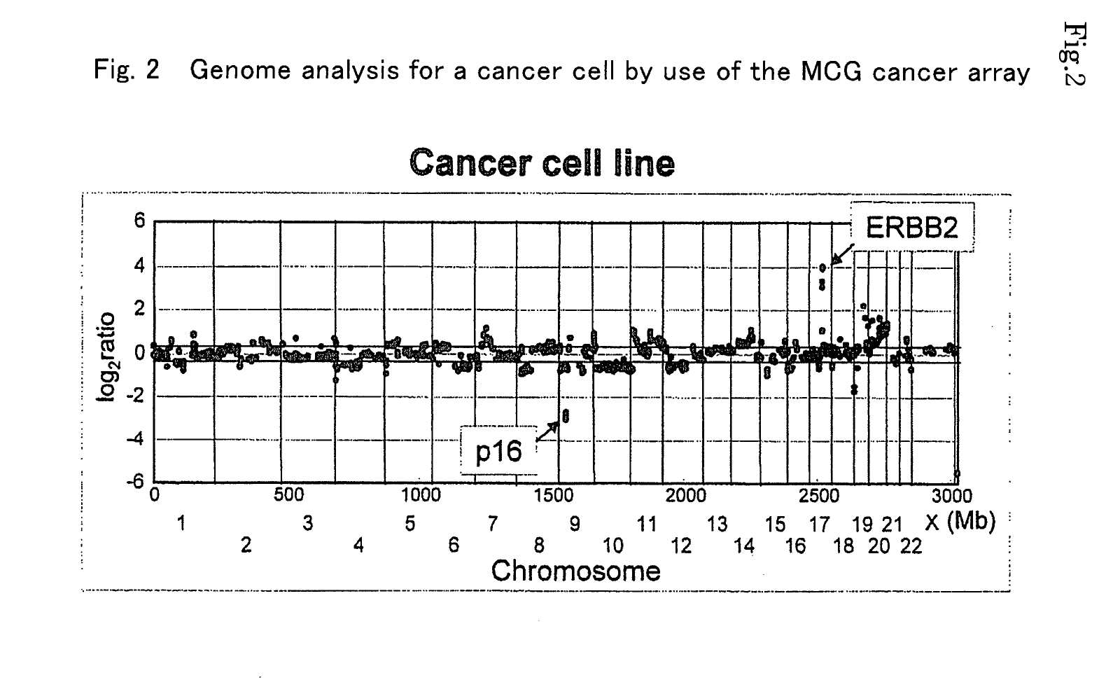

Furthermore, actually, DNA derived from a normal cell was labeled with Cy-5 and DNA derived from a cancer cell was labeled with Cy-3. They were subjected to comparative genomic hybridization. Data were taken in by a GenePix 4000B scanner. Individual pixels were analyzed and the results are shown in FIG. 2. The vertical axis of the graph in FIG. 2 is indicated by Log₂ Ratio and BAC clones having genomes from a short arm to a long arm of a chromosome are arranged on the transverse axis. The Cy-3 intensities of all spots are corrected to the same level as the Cy-5 intensities of all spots, and the ratio of Cy-3 intensity/Cy-5 intensity of each spot is obtained and a value of Log₂Ratio is computationally obtained. BAC having a CDKN2A (p16) gene shows Log₂Ratio=about −3 and Ratio=1/8, which clearly indicates a homozygous deletion. On the other hand, BAC having ERBB2 gene gives Log₂Ratio=3-4 and Ratio=8-16, which demonstrates that ERBB2 genomic DNA is amplified 8 to 16 fold.

To identify a group of genes present in the chromosomal region amplified and deleted in a cancer cell by use of the MCG cancer array, genomic DNA derived from a healthy person and genomic DNA derived from a lung cancer cell are labeled with mutually different dye, for example, Cy-3 and Cy-5, in accordance with a customary method (for example, a nick translation method using dCTP). The labeling kits using the nick translation method using dCTP are sold by PanVera (Takara Shuzo Co., Ltd., a distributor in Japan) and Invitrogen (CA, USA). When the labeled DNA is hybridized with the DNA printed on the CGH array, it is more preferable to add Cot-1DNA, formamide, dextran sulfate, SSC (150 mM NaCl/15 mM sodium citrate), Yeast t-RNA, and SDS (sodium dodecyl sulfate). Furthermore, it is preferable to add a solution containing labeled DNA after it is denatured with heat. As a container for use in hybridization, a container that can be placed on a platform having a locking function and can bring a small amount of solution uniformly into contact with the array is preferable, and use of e.g., hybriman, is more preferable. The temperature of hybridization is preferably 30 to 70° C. and more preferably 38 to 45° C. The hybridization time is preferably 12 to 200 hours and more preferably 40 to 80 hours. The array can be washed with formamide, SSC solution or the like at room temperature. The washing of the array is an important step to reduce a nonspecific signal as much as possible. More preferably, the array was washed at room temperature, and then, washed with the same washing solution at 40 to 60° C., further washed in a solution containing SSC-SDS at 50° C., allowed to stand in a solution containing phosphate buffer/NP-40, and finally shaken in a solution containing SSC.

(1) Group of Genes Present in the Chromosome Amplified and Deleted in a Lung Cancer Cell

Using the MCG cancer array, a group of genes on the chromosome of lung squamous cell carcinoma, lung adenocarcinoma or lung large cell carcinoma was checked. More specifically, a gene having a Ratio value of 1.32 or more, suggesting that the gene has not less than 3 amplified copies (normally having 2 amplified copies), present on the chromosome, was checked. As a result, MUC1gene, PRCC gene, EIF4G gene, THPO gene, TERT gene, DAB2gene, EGFR gene, ELN gene, MUC3 gene, MYC gene, PVT1gene, FACA gene, PTPN1 gene and Livin gene were detected. Furthermore, as a gene present in the chromosome and having a Ratio value of not less than 4, that is, having not less than 8 amplified copies (normally 2 copies are present), MYCN gene, MYC gene, PVT1 gene, ELK3 gene, BCL2L2 gene, HNF3A gene, ERBB2 gene and CGI-147 gene were detected.

As a gene on the heterozygote chromosome and having a Ratio value of 0.75 or less, that is, an amplified copy number is reduced by half, BAIAP1 gene, LZTS1 gene, AAC1 gene, BLK gene, MTAP gene, CDKN2A(p16) gene, GPC5 gene, SNRPN gene, SAMD4 gene, DCC gene, and ADRBK2gene were detected. Furthermore, as a gene on the chromosome having a Ratio value of 0.25 or less, that is, having a homozygous deletion, CDKN2A(p16) gene, MTAP gene, DBCCR1 gene and RIZ gene were detected. In the case of DBCCR1 gene, suppression of expression due to a homozygous deletion and suppression of expression due to methylation of a CpG island were found.

Lung squamous cell carcinoma, lung adenocarcinoma or lung large cell carcinoma can be diagnosed by monitoring amplification and deletion of a group of genes thus detected.

A group of genes of amplified and deleted chromosomal region in a non-small cell lung cancer cell was analyzed in the same manner as above. As a result that a group of genes having a Ratio value of 1.32 or more, that is, having not less than 3 copies (although normally having 2 copies) was checked, ESP15, MUC1, ARHGEF2, PRCC, ABCC5, EIF4G1, CDH12, PC4, SKP2, DAB2, ZNF131, RAD52, WNT3, CDC27, COL1A1, ABCC3, PPMID, ITGB4, BIRC5, SHGC-103396, BCL2L1, RBL1, SRC, TGIF2, MYBL2, BIRC7, ARAF1, CUL4B, and CTAG1 were detected. As a gene having a Ratio value of 4 or more, that is, having not less than 8 amplified copies (although normally having 2 copies), TRIM33, MYCL1, RLF, MYC, CCNE1, and PCTK1 genes were detected.

As a gene of a heterozygote having a Ratio value of 0.75 or less, that is, having a copy number reduced by half, CCK, UBE2E1, THRB, RARB, TGFβR2, MLH1, CTNNB1, VIPR1, ZNF35, TGM4, TDGF1, PTPRG, FHIT, BAIAP1, MITF, LOC151987, EIF4E, NFκB, CCNA, PGRMC2, VEGFC, MSH3, RASA1, TPT1, RB1, AATF, and ADRBK2 were detected. Furthermore, as a gene having a Ratio value of 0.25 or less, having a heterozygous deletion, NR2F1 gene was found.

Non-small cell lung cancer can be diagnosed by monitoring an amplification or deletion of a chromosomal region having a group of genes thus detected.

As described above, the amplification and deletion of the chromosomal region in lung cancer are analyzed by use of the MCG cancer array, and thus a group of genes having amplified and deleted can be identified. Based on the results, it is possible to understand the state of each cancer. To describe more specifically, it is possible to determine whether a tumor is benign, intermediate or malignant. In the case of a malignant tumor, it is possible to provide important findings to determine the grade of the cancer. It is further possible to provide data for efficient chemotherapy performed after a cancerous foci is surgically removed.

As described above, in the DBCCR1 gene, gene expression did not take place not only due to a homozygote deletion but also due to methylation of a CpG island of the gene not deleted. For these reasons, the probability that expression of DBCCR1 gene is suppressed is high in non-small cell lung cancer.

It is possible and preferable to simultaneously detect deletion of a chromosome and suppression of expression by monitoring the gene expression by a real time RT-PCR method or a DNA chip method in a deletion cancer gene group.

B. Suppression/Treatment Means for a Cancer by a Cancer-Associated Gene

The suppression/treatment means for a cancer provided by the present invention are roughly divided into two groups. One (1) is a method of suppressing the cancer cell (hereinafter referred to as “suppression/treatment means 1”) by introducing a gene whose deletion is associated with canceration of a cell (called as a deletion cancer gene) into a cancer cell. The other (2) is a method of suppressing the cancer cell (hereinafter referred to as “suppression/treatment means 2”) by applying a nucleic acid antagonizing against a transcriptional product of a gene whose amplification is associated with canceration of a cell (called as an amplification cancer gene) to a cancer cell.

(1) Suppression/Treatment Means 1

Of the deletion cancer genes mentioned above, many of the genes in the chromosomal region exhibiting a homozygous deletion are detected to fall within the category of a cancer suppressor gene. Of them, a gene suppressing proliferation of target cancer cells or a gene inducing apoptosis of cancer to death can be introduced into a cancer cell by use of a Sendai virus vector or adenovirus vector. In a gene therapy using these virus vectors, as a promoter for the homozygous deletion gene to be expressed, a promoter highly expressed in a cancer tissue but not highly expressed in a normal tissue, such as human CXCR4 promoter (Zhu Z B, Makhija S K, Lu B, Wang M, Kaliberova L, Liu B, Rivera A A, Nettelbeck D M, Mahasreshti P J, Leath C A, Yamaoto M, Alvarez R D, Curiel D T: Transcriptional targeting of adenoviral vector through the CXCR4 tumor-specific promoter, Gene ther., 11, 645-648, 2004) and Survivin promoter are preferably used. Each of these recombinant viruses can be combined with a ribosome to form a composite, which may be introduced into a cancer tissue. Alternatively, it can be introduced in the form of naked DNA into a cancer tissue.

Using a viral vector and a promoter as mentioned above, each cancer therapy can be made by selecting a gene from following candidate genes:
MTAP gene and CDKN2A (p16) gene localized in 9p21 and DBCCR1 gene localized in 9p34, and RIZ gene localized in 1p36, for a non-small cell lung cancer (squamous cell carcinoma, adenocarcinoma, large cell carcinoma); and NR2F1gene localized in 5q15 for a small cell lung cancer.

CDKN2A (p16) gene is a cyclin dependent kinase inhibitor located in a chromosome 9p21 and considered as a cancer suppressor gene. P16 protein, when it binds to CDK4 kinase, is suppressed in its activity, thereby suppressing cell cycle progression. The CDKN2A (p16) gene is deleted in a wide variety of cancers such as acellular esophageal carcinoma, malignant glioma, gastric carcinoma, pancreatic carcinoma and thyroid carcinoma. MTAP is a gene encoding 5′-methylthioadenosinephosphorylase, which is the first enzyme of a methionine salvage pathway and considered as a cancer suppressor gene. The product of the methionine salvage pathway inhibits the activity of ornithine decarboxylase highly expressed in cancer. RIZ is a gene encoding an RB interacting Zinc Finger protein found in leukemia and belongs to Nuclear protein methyltransferase superfamily. DBCCR1 is found as a gene deleted in chromosome 1 of the bladder carcinoma and considered as a cancer suppressor gene. TEK is an angiopoietin-1 receptor, which is otherwise designated as Tie-2. When TEK is phosphorylated by tyrosine kinase, angiogenesis is induced. CDH23 is cadherin related 23 gene, belongs in the cadherin superfamily, and is a glycoprotein associated with calcium dependent cell adhesion. CXADR gene encodes receptors of coxsachie virus and adenovirus. cIAP1 gene encodes an apoptosis inhibitor. FLI1 gene is classified into an ETS transcription factor. TSPY gene is present in human Y chromosome and encodes a testis specific protein. LRP1B is abbreviation of lipoprotein receptor-related protein 1B, which is a cellular membrane receptor using urokinase and a plasminogen activator, etc., as a ligand, and is considered as a cancer suppressor gene. DEC1 refers to “deleted in esophageal cancer 1” and loss of heterozygosity is frequently detected in esophageal carcinoma and squamous cell carcinoma of the bladder, lung and head and neck portion. MMP1 and MMP7 are matrix metalloproteinase and enzymes involved in vascularization. SMAD4 gene is a cancer suppressor gene whose deletion is found in pancreatic carcinoma and encodes a protein that is activated by a receptor and transferred to a nucleus to derive a transcriptional activation activity. ETS1 is a transcription factor and derives angiopoietin-2 gene, etc. RB1 is a retinoblastoma gene and a cancer suppressor gene.

A virus vector is prepared by integrating a gene as mentioned above downstream of a promoter highly expressed in a cancer tissue, and is then introduced into the cancer tissue of a cancer patient. The gene is allowed to express, thereby reducing cancer in size and inhibiting metastasis. In this way, recurrence of cancer after cancer is excised out can be prevented.

(2) Suppression/Therapeutic Means 2

Of the amplification cancer genes found above, a group of genes present in the chromosome, amplified 4-fold or more than that of a normal cell, are shown in Table 1.

TABLE 1

| Type of | | | | | | |
|---|---|---|---|---|---|---|
| cancer cell | Name of amplified gene | | | | | |
| SCLC | MYCN | MYC | PVT1 | ELK3 | BCL2L2 | HNF3A |
| | ERBB2 | CGI-147 | | | | |
| NSCLC | TRIM33 | MYCL1 | RLF | MYC | CCNE1 | PCTK1 |

When these groups of genes are compared to those of a normal cell, the number of genome copies in chromosomes 1 to 22 increases to 8 or more, and that in X and Y chromosomes increases 4 or more. The transcriptional product of a highly expressed gene is decomposed by adding the small interference RNA corresponding to the transcriptional product (mRNA) in accordance with an RNAi (RNA interference) method. In this manner, cancer can be treated. Design and synthesis of siRNA and the transfection of siRNA to a cell, confirmation of the effect of RNAi can be performed by conventional methods with reference to, for example, Takara Bio RNAi Book, “Experimentation protocol” (published by Takara Bio Inc., Shiga prefecture). Examples of siRNA to be used herein include Hairpin siRNA, which can be expressed by using an siRNA oligonucleotide and a pSilencer vector (manufactured by Funakoshi Co., Ltd., Tokyo).

On the other hand, mRNA of a cancer gene amplified and excessively expressed in a cancer can be knocked out by use of an antisense oligonucleotide. In this case, s-oligonucleotide is preferably used to inhibit amplification of a cancer cell since it has a good intracellular stability compared to a general oligonucleotide. SiRNA, Hairpin siRNA and s-oligonucleotide, which are found to be effective by use of a cancer cell, can be evaluated in a nude mouse having a cancer cell transplanted therein.

In this case, it is preferable to construct a delivery system such that these RNA can be accumulated in a cancer tissue.

### EXAMPLES

### Example 1

### Preparation of “MCG Cancer Array”

Based on the search for genome database website of the National Cancer for Biotechnology and University of California, Santa Cruz Biotechnology as well as BLAST search of DNA screened, BAC/PAC clones having an extremely important gene for canceration and amplification of a cancer cell or having a sequence tagged site marker were selected.

BAC and PAC DNA was digested with Dpnl, RsaI, and HaeIII, and thereafter ligated with adaptor DNA. PCR was performed twice using a primer having the sequence of the adaptor. One of the two ends of the primers has the 5′ end aminated. This process is called an inexhaustible process and DNA thus obtained is defined as inexhaustible DNA. The inexhaustible DNA is placed in an ink-jet type spotter (GENESHOT, NGK Insulators, Ltd., Nagoya) and covalently printed, in duplicate, onto an oligo DNA micro array (manufactured by Matsunami Glass, Osaka).

### Example 2

### Collective Analysis of a Cancer-Associated Gene in Non-Small Cell Lung Cancer by Use of MCG Cancer Array

Using the “MCG cancer array,” a gene amplified or deleted was checked with respect to 27 types of non-small cell lung cancer strains, which consisted of squamous cell carcinoma (11 strains), adenocarcinoma (10 strains), and large cell carcinoma (6 strains). Genomic DNA samples were prepared from these cells. As a control, a genomic DNA sample taken from a healthy male was used. A genomic DNA sample (0.5 mg) was digested with DpnI and labeled by nick translation in the presence of 0.2 mM dATP, 0.2 mM dTTP, 0.2 mM dGTP, 0.1 mM dCTP and 0.4 mM Cy3-dCTP (lung cancer cell) or 0.4 mM Cy5-dCTP (normal cell). Cy3- and Cy5-labelled dCTP were obtained from Amersham Biosciences (Tokyo). The genomic DNA labeled with both labels was precipitated by adding ethanol in the presence of Cot-1 DNA (Invitrogen) and dissolved in 120 μl of hybridization solution mixture (50% formamide 10% dextran sulfate, 2×SSC (1×SSC: 150 mM Nacl/15 mM sodium citrate), 4% sodium dodecylsulfate, pH7). After incubation at 37° C. for 30 minutes, the mixture was added to the CGH array set in a hybridization chamber. Incubation was performed at 37° C. while shaking at a speed of 3 rpm (round per minute) for 48 to 72 hours. Thereafter, the CGH array was washed with a 50% formamide/2×SSC (pH 7.0) solution at 50° C. for 15 minutes, next by 2×SSC/0.1% SDS at 50° C. for 15 minutes, and further by 0.1 M phosphate buffer solution (pH8) containing 0.1% Nonidet P-40 at room temperature for 15 minutes. After dried in air, the CGH array was monitored by GenePix 4000 B scanner (Axon Instruments, CA, USA) to check fluorescence derived from Cy3 and Cy5 (FIG. 1). The results thus obtained were analyzed by Genepix Pro 4.1 imaging software (Axon Instruments). The ratio Cy3/Cy5 was obtained by adjusting the average of fluorescent intensity derived from Cy3 and that derived from Cy5 to the same value. When there is no abnormality in the genome, the ratio is 1. Determination was made as follows. When the Ratio value is 1.32 or more, amplification takes place in the genome and when the Ratio value is 4 or more, significant amplification takes place in the genome. When the Ratio value is 0.75 or less, there is a possibility of a heterozygous deletion in the genome, and when the Ratio value is 0.25 or less, there is an extremely large possibility of a homozygous deletion.

As shown in Table 2, as the gene having a Ratio value of 1.32 or more (Log₂Ratio is 0.4 or more) in squamous cell carcinoma (SCC cells), adenocarcinoma (AdC cells) and large cell carcinoma (LCC cells), MUC1 gene, PRCC gene, EIF4G gene, THPO gene, TERT gene, DAB2 gene, EGFR gene, ELN gene, MUC3 gene, MYC gene, PVT1 gene, FACA gene, PTPN1 gene and Livin gene were detected.

On the other hand, as an amplified gene having a Ratio value of 4 or more (Log₂Ratio is 2 or more), MYCN gene localized in chromosome 2p24 (1 cell line), MET gene localized in chromosome 7q23 (2 cell lines), MYC gene localized in chromosome 8q24 (4 cell lines), PVT1 gene localized in chromosome 8q24 (4 cell lines), ELK3 gene localized in chromosome 12q23 (1 cell line), BCL2L2 gene localized in chromosome 14q11(1 cell line), HNF3A gene localized in chromosome 14q12 (1 cell line), ERBB2 gene localized in chromosome 17q12 (1 cell line), and CGI-147 gene localized in chromosome 17q22 (1 cell line) were detected.

TABLE 2
Name of gene amplified and having a Ratio value of
1.32 or more in non-small cell lung cancer cell

| | | Cell line in which gene | | | |
|---|---|---|---|---|---|
| | Name of | amplification was detected | | | |
| Chromosomal | amplified | Per- | | | |
| region | gene | centage | SCC cell | AdC cell | LCC cell |
| 1q21 | MUC1 | 33% | 3/11 cells | 5/10 cells | 1/6 cells |
| 1q21 | PRCC | 37% | 3/11 cells | 4/10 cells | 3/6 cells |
| 3q25-tel | EIF4G | 44% | 6/11 cells | 6/10 cells | 0/6 cells |
| 3q25-tel | THPO | 33% | 6/11 cells | 3/10 cells | 0/6 cells |
| 5p12-15 | TERT | 48% | 7/11 cells | 5/10 cells | 1/6 cells |
| 5p12-15 | DAB2 | 44% | 7/11 cells | 4/10 cells | 1/6 cells |
| 7p12-15 | EGFR | 41% | 7/11 cells | 3/10 cells | 1/6 cells |
| 7q11-31 | ELN | 33% | 5/11 cells | 4/10 cells | 0/6 cells |
| 7q11-31 | MUC3 | 44% | 6/11 cells | 6/10 cells | 0/6 cells |
| 8q24 | MYC | 52% | 5/11 cells | 5/10 cells | 4/6 cells |
| 8q24 | PVT1 | 56% | 5/11 cells | 6/10 cells | 4/6 cells |
| 16q24 | FACA | 33% | 5/11 cells | 2/10 cells | 2/6 cells |
| 20q11-13 | PTPN1 | 33% | 6/11 cells | 1/10 cells | 2/6 cells |
| 20q11-13 | Livin | 33% | 6/11 cells | 2/10 cells | 1/6 cells |

Percentage is percent (%) based on the total non small lung cancer cell lines tested. SCC cells, AdC cells and LCC cells are abbreviations of squamous cell carcinoma, adenocarcinoma, and large cell carcinoma, respectively. The number of cell lines is shown in which a gene amplification was detected.

As shown in Table 3, as a gene whose copy number is reduced by half (heterozygote) and having a Ratio value of 0.75 or less (Log₂Ratio is −0.4 or less) in squamous cell carcinoma, adenocarcinoma, and large cell carcinoma, BAIAP1 gene, LZTS1 gene, AAC1 gene, BLK gene, MTAP gene, p16 gene, GPC5 gene, SNRPN gene, SAMD4 gene, DCC gene, and ADRBK2 gene were found. Furthermore, as a gene having a Ratio value of 0.25 or less (Log₂Ratio is −2.0 or less) and having a homozygous deletion, CDKN2A (p16) gene present in chromosome 9p21 (6 cell lines), MTAP gene present in chromosome 9p21 (4 cell lines), RIZ gene present in chromosome 1p36 (1 cell line), and DBCCR1 gene presenting chromosome 9p34 (1 cell line) were detected.

TABLE 3
Name of gene deleted and having a Ratio value reduced
to 0.75 or less in non-small cell lung cancer cell

| | | Cell line in which gene | | | |
|---|---|---|---|---|---|
| | Name of | deletion was detected | | | |
| Chromosomal | amplified | Per- | | | |
| region | gene | centage | SCC cell | AdC cell | LCC cell |
| 3p11-14 | BAIAP1 | 48% | 7/11 celsl | 5/10 cells | 1/6 cells |
| 8p21-tel | LZTS1 | 58% | 7/11 cells | 5/10 cells | 3/6 cells |
| 8p21-tel | AAC1 | 56% | 6/11 cells | 7/10 cells | 2/6 cells |
| 8p21-tel | BLK | 59% | 6/11 cells | 6/10 cells | 4/6 cells |
| 9p21-22 | MTAP | 56% | 7/11 cells | 5/10 cells | 3/6 cells |
| 9p21-22 | CDKN2A | 70% | 8/11 cells | 6/10 cells | 4/6 cells |
| | (p16) | | | | |
| 13q22-tel | GPC5 | 41% | 4/11 cells | 7/10 cells | 0/6 cells |
| 15q12 | SNRPN | 44% | 4/11 cells | 4/10 cells | 4/6 cells |
| 18q21 | SMAD4 | 44% | 7/11 cells | 3/10 cells | 2/6 cells |
| 18q21 | DCC | 44% | 6/11 cells | 4/10 cells | 2/6 cells |
| 22q | ADRBK2 | 44% | 4/11 cells | 5/10 cells | 2/6 cells |

Percentage is percent (%) based on the total non small lung cancer cell lines tested. SCC cells, AdC cells and LCC cells are abbreviations of squamous cell carcinoma, adenocarcinoma, and large cell carcinoma, respectively. The number of cell lines is shown in which a gene amplification was detected.

### Example 3

### Collective Analysis of a Cancer-Associated Gene in Small Cell Lung Cancer (SCLC) by Use of MCG Cancer Array

Using the “MCG cancer array,” gene amplification in a target small-cell lung cancer was analyzed. The results are shown in Table 4. In about a half of the cell lines tested, ESP15, MUC1 , ARHGEF2, PRCC, ABCC5, EIF4G1, CDH12, PC4, SKP2, DAB2, ZNF131, RAD52, WNT3, CDC27, COL1A1, ABCC3, PPM1D, ITGB4, BIRC5, SHGC-103396, BCL2L1, RBL1, SRC, TGIF2, MYBL2, BIRC7, ARAF1, CUL4B, CTAG1 genes were amplified and had a Ratio value of 1.32 or more.

TABLE 4
Name of gene amplified and having a Ratio value
of 1.32 or more in small cell lung cancer cell

| | | Cell line in which gene | |
|---|---|---|---|
| Chromosomal | Name of | amplification was detected | |
| region | amplified gene | Number of cell lines | Percentage |
| 1p32.3 | ESP15 | 9 | 41% |
| 1q22 | MUC1 | 11 | 50% |
| 1q22 | ARHGEF2 | 9 | 41% |
| 1q23.1 | PRCC | 10 | 46% |
| 3q27.1 | ABCC5 | 11 | 50% |
| 3q27.1 | EIF4G1 | 10 | 46% |
| 5p14.3 | CDH12 | 11 | 50% |
| 5p13.3 | PC4 | 11 | 50% |
| 5p13.2 | SKP2 | 11 | 50% |
| 5p13.1 | DAB2 | 9 | 41% |
| 5p12 | ZNF131 | 9 | 41% |
| 12p13.33 | RAD52 | 10 | 46% |
| 17q21.32 | WNT3, CDC27 | 11 | 50% |
| 17q21.33 | COL1A1, ABCC3 | 9 | 41% |
| 17q23.2 | PPM1D | 9 | 41% |
| 17q25.1 | ITGB4 | 9 | 41% |
| 17q25.3 | BIRC5 | 10 | 46% |
| 17q25.3 | SHGC-103396 | 9 | 41% |
| 20q11.21 | BCL2L1 | 9 | 41% |
| 20q11.23 | RBL1, SRC | 9 | 41% |
| 20q11.23 | TGIF2 | 10 | 46% |
| 20q13.12 | MYBL2 | 9 | 41% |
| 20q 13.3 | BIRC7 | 9 | 41% |
| Xp11.3 | ARAF1 | 9 | 41% |
| Xq24 | CUL4B | 11 | 50% |
| Xq28 | CTAG1 | 11 | 50% |

As shown in Table 5, as a gene having a Ratio value of 4 or more, that is, a gene amplified not less than 4-fold compared to the gene in a normal cell, TRIM33, MYCL1, RLF, MYC, CCNE1, and PCTK1 genes were found.

TABLE 5
Name of gene in which amplification was detected
and having a Ratio value of 4 or more (high level
amplification) in small cell lung cancer cell

| Chromosomal region | Name of amplified gene | Number of cell lines |
|---|---|---|
| 1p13.2 | TRIM33 | 1 |
| 1p34.2 | MYCL1, RLF | 2 |
| 8q24.21 | MYC | 2 |
| 19q12 | CCNE1 | 1 |
| Xp11.3 | PCTK1 | 1 |

Next, as a gene having a Ratio value reduced to 0.75 or less in a non-small lung cancer cell, that is, a gene determined as heterozygote, CCK, UBE2E1, THRB, RARB, TGFβR2, MLH1, CTNNB1, VIPR1, ZNF35, TGM4, TDGF1, PTPRG, FHIT, BAIAP1, MITF, LOC151987, EIF4E, NFκB, CCNA, PGRMC2, VEGFC, MSH3, RASA1, TPT1, RB1, AATF, and ADRBK2 genes were detected (Table 6).

TABLE 6
Name of gene having a Ratio value of 0.75
or less in non-small cell lung cancer cell

| | | Cell line in which gene | |
|---|---|---|---|
| Chromosomal | Name of | deletion was detected | |
| region | amplified gene | Number of cell lines | Percentage |
| 3p (random) | CCK | 15 | 68% |
| 3q24.2 | UBE2E1, THRB | 14 | 64% |
| 3p24.2 | RARB | 15 | 68% |
| 3q24.1 | TGFBR2 | 14 | 64% |
| 3p22.3 | MLH1 | 14 | 64% |
| 3p22.1 | CTNNB1 | 15 | 68% |
| 3p22.1 | VIPR1 | 12 | 55% |
| 3p21.32 | ZNF35 | 16 | 72% |
| 3p21.31 | TGM4 | 13 | 59% |
| 3p21.31 | TDGF1 | 16 | 72% |
| 3p14.2 | PTPRG | 16 | 72% |
| 3p14.2 | FHIT | 14 | 64% |
| 3p14.1 | BAIAP1 | 16 | 72% |
| 3p13 | MITF, LOC151987 | 16 | 72% |
| 4q23 | EIF4E | 12 | 55% |
| 4q24 | NFκB | 12 | 55% |
| 4q27 | CCNA | 12 | 55% |
| 4q28.2 | PQRMC2 | 12 | 55% |
| 4q34.3 | VEGFC | 12 | 55% |
| 5q14.1 | MSH3 | 12 | 55% |
| 5q14.3 | RASA1 | 12 | 55% |
| 13q14.13 | TPT1 | 12 | 55% |
| 13q14.2 | RB1 | 13 | 59% |
| 17q12 | AATF | 12 | 55% |
| 22q12.1 | ADRBK2 | 12 | 55% |

Furthermore, as a gene having a Ratio value reduced to 0.25 or less, that is, a gene having a homozygous deletion, NR2F1 gene was found (Table 7). A group of genes having heterozygote and homozygote significantly decreases in expression level, which may possibly be a cause of cancer.

TABLE 7
Name of gene having a Ratio value reduced to
0.25 or less in small cell lung cancer cell

| Chromosomal region | Name of amplified gene | Number of cell lines |
|---|---|---|
| 5q15 | NR2F1 | 1 |

### Example 4

### Inhibition of Proliferation of Squamous Cell Sarcoma and Treatment of Nude Mouse Carrying a Cancer by Infection of Sendai Virus Having CDKN2A (p16) Gene Integrated Therein

CDKN2A (p16) gene was ligated downstream Survivin promoter and integrated into a Sendai virus vector. The resultant viral DNA was packaged to produce a recombinant virus. The recombinant virus was purified by discontinuous iodixanol gradient centrifugation and a heparin agarose column. Squamous cell carcinoma was inoculated in a 96 well tissue culture plate at a concentration of 5×10³ cell/well and incubated in a CO₂ incubator at 37° C. for one day. Then, 100 moi of purified recombinant Sendai virus was infected per well and incubated for 72 hours. The amplification level of cells was determined by a 3-(4,5-dimethylthiazol-2-yl)-2,5-diphenyl tetrazolium bromide assay using a commercially available kit (manufactured by Promega, Tokyo) in accordance with the instruction. As a result, in a cell infected with the virus having CDKN2A (p16) gene integrated therein, significant inhibition of proliferation was observed. The cell extraction solution was subjected to Western blot analysis. CDKN2A (p16) protein was not detected in a control Sendai virus infection cell, whereas a clear band of CDKN2A (p16) protein was detected from the cell extraction solution sample. From the results, it was demonstrated that proliferation of carcinoma cells is suppressed by infecting the cells with Sendai virus having CDKN2A (p16) gene integrated therein. This means that Sendai virus having CDKN2A (p16) gene integrated therein can be used for reducing carcinoma or suppressing minute metastasis.

Next, a nude mouse was inoculated with squamous cell carcinoma (cells), and simultaneously, infected with 3×10¹¹ moi of purified recombinant Sendai virus. Inhibition of carcinoma proliferation was monitored while expecting an increase of the life time of the mouse, in other words, an increase of efficiency of the gene therapy according to the present invention.

Based on the results, a clinical trial of gene therapy for a human patient can be planned.

### Example 5

### Inhibition of Proliferation of Small-Cell Lung Cancer Cell by an SKP2 Gene Antisense Oligonucleotide

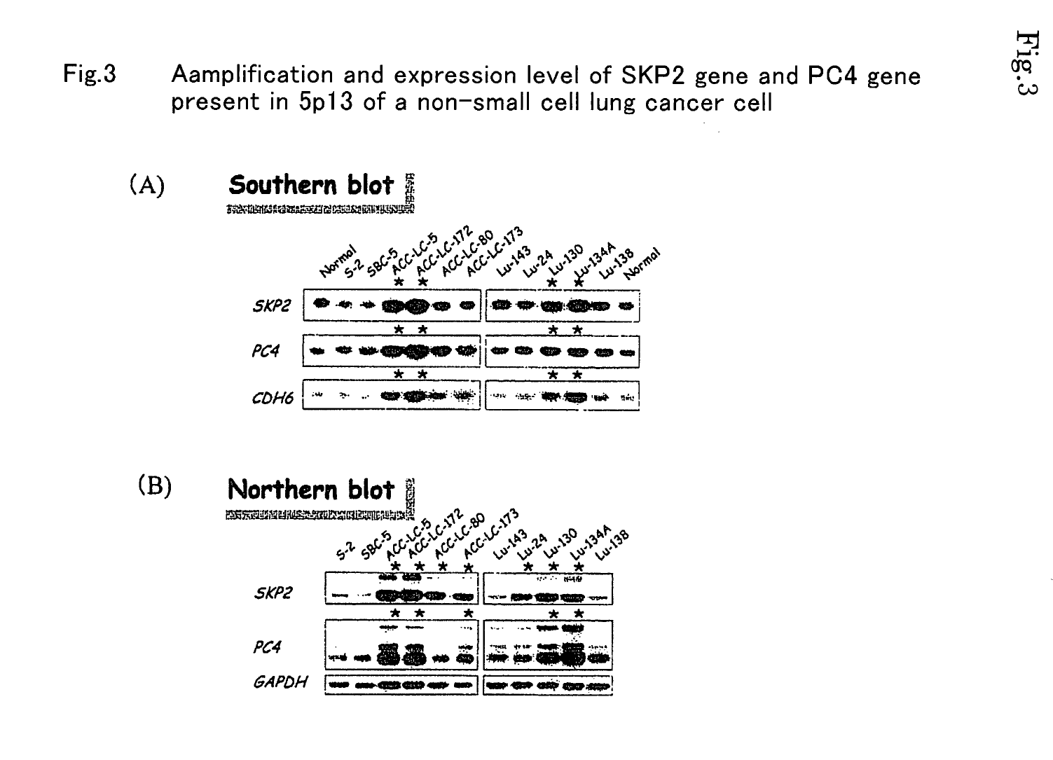

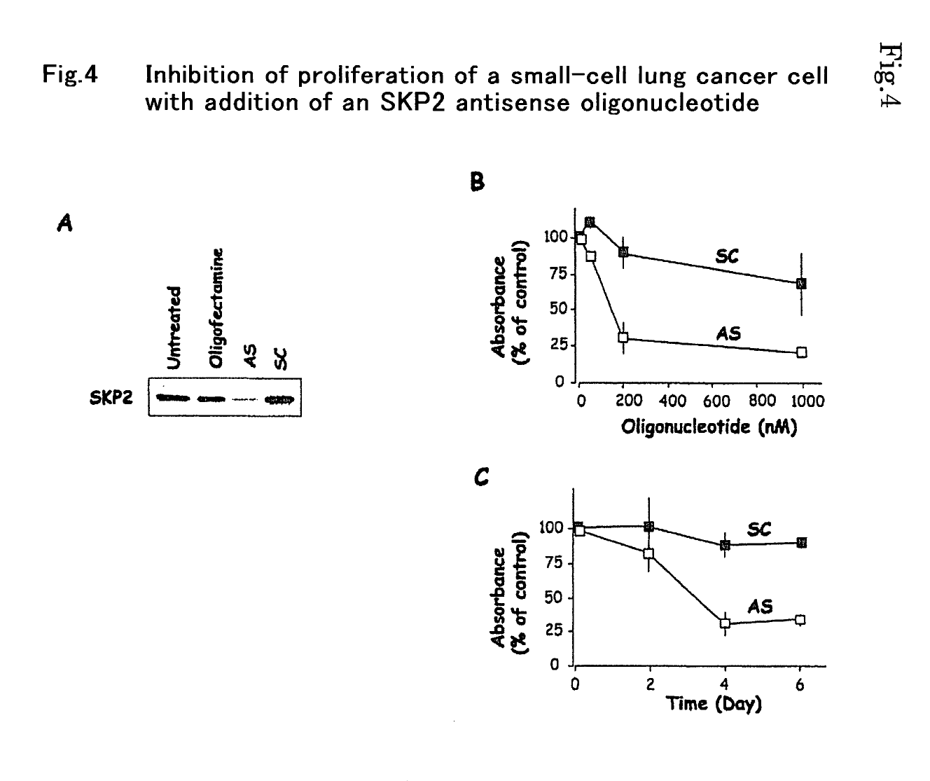

In small cell lung cancer cell, a chromosomal 5p13 region is amplified. Of the small cell lung cancer cell lines, ACC-LC-5 cell line, ACC-LC-172 cell line, Lu-130 cell line, and Lu-134 cell line were investigated. As a result, CDH6, PC4, and SKP2 genes present in the 5p13 region were significantly amplified at a chromosome level by the Southern blot method. When the expression of these genes was analyzed by the Northern blot method, it was found that a significant increase was observed compared to a normal cell (FIG. 3). As a result of culturing these cells in the presence of an SKP2 antisense oligonucleotide, the amount of SKP2-mRNA was significantly reduced. In accordance with this, the proliferation of cells was suppressed to a level of 25% compared to a control cell to which a sense oligonucleotide was added. The inhibition with the SKP2 antisense oligonucleotide added to the cell reached a maximum at a concentration of 200 nM to 1 μM. Cell proliferation reduced to a level of 25% 4 days after initiation of the proliferation (FIG. 4).

From cells not treated (untreated), cells treated only with a transfection reagent (oligofectamine), cells transfected with an SKP2 antisense oligonucleotide (AS) and cells transfected with a SKP2 sense oligonucleotide (SC), RNA was prepared, and the expression level of SKP2 mRNA was analyzed by the Northern blot method. The results are shown in FIG. 4(A). It was found that the SKP2 mRNA expression is significantly reduced by the treatment of AS.

Inhibition of proliferation of small cell lung cancer cells by the SKP2 antisense oligonucleotide was investigated. The results are shown in FIG. 4(B). The vertical axis indicates cell proliferation, which is indicated by a relative percentage based on the proliferation of untreated cells (100%). Proliferation was investigated by adding the SKP2 sense oligonucleotide and the SKP2 antisense oligonucleotide in the range of 0 to 1000 nM.

FIG. 4(C) shows a change of proliferation of small cell lung cancer cells by the SKP2 antisense oligonucleotide over time. The vertical axis is the same as in FIG. 4(B). The transverse axis indicates the number of culture days after the SKP2 sense oligonucleotide and SKP2 antisense oligonucleotide are added.

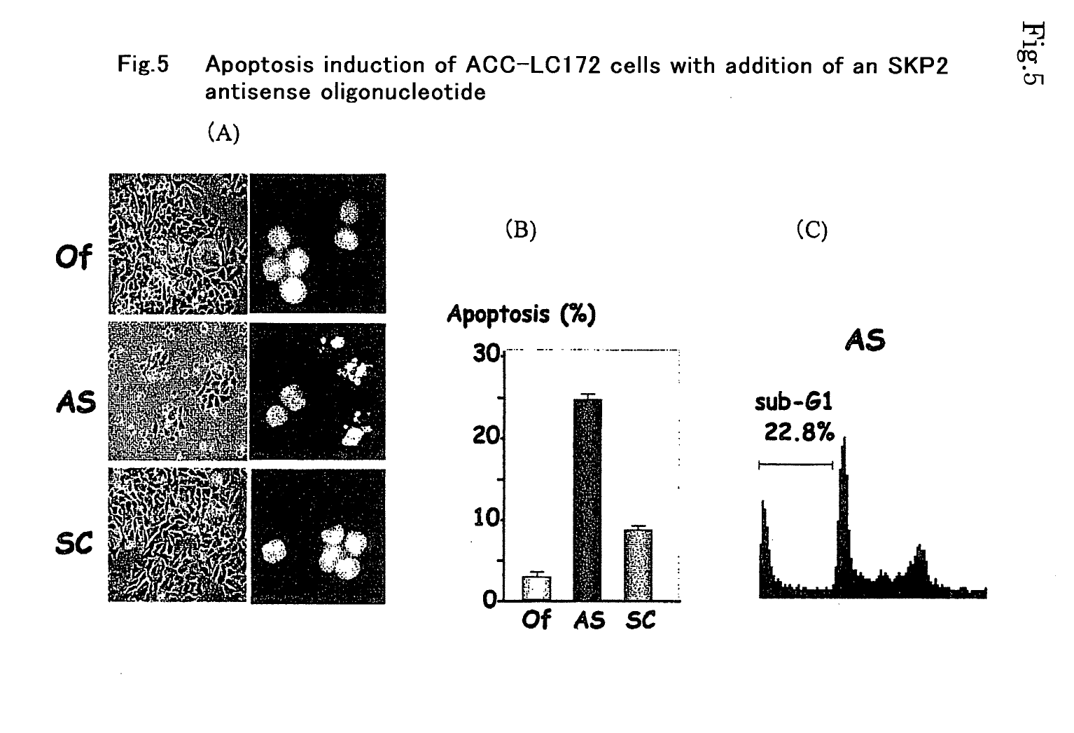

FIG. 5 shows induction of apoptosis of ACC-LC172 cells by addition of the SKP2 antisense oligonucleotide. As is demonstrated in FIG. 5, the number of ACC-LC172 cells causing apoptosis to death by addition of the SKP2 antisense oligonucleotide increases to 25% from 3% of non-treated cells (see FIG. 5B). Apoptosis was confirmed by morphological observation and flow cytometric analysis.

Note that FIG. 5A shows microphotographs of control cells (Of), cells (AS) transfected with the SK2P antisense oligonucleotide, and cells (SC) transfected with the SKP2 sense oligonucleotide. AS shows typical results of cells causing apoptosis. FIG. 5B shows the percentage (vertical axis) of cells causing apoptosis. Reference symbols Of, AS and SC indicate the same as in FIG. 5A. It is found that apoptosis is noticeably induced by the SKP2 antisense oligonucleotide. FIG. 5C shows the results of flow cytometry of ACC-LC172 cells treated with AS. Typical results of apoptosis are shown also herein.

These results demonstrate that the SKP2 antisense oligonucleotide specifically inhibits proliferation of cancer cells. It is therefore apparent that the SKP2 antisense oligonucleotide can be used as a therapeutic agent for cancer.

### Example 6

### Reduction in Expression of DBCCR1 Gene by Methylation in Non-Small Cell Lung Cancer Cell

Eight non-small cell lung cancer cell lines were checked for deletion of DBCCR1 gene by genomic PCR. As a result, only one cell showed a homozygous deletion and the remaining 7 cells had 2 copies. Next, when the expression of DBCCR1 gene was analyzed by the RT-PCR method, the expression was not detected in the all 8 cells. Then, degree of methylation was investigated by a bisulfite sequence method with respect to 7 cell lines in which DBCCR1 gene was not deleted and no expression of the gene was detected. The gene was methylated in the all 7 cell lines. From this, it was found that the expression of DBCCR1 gene is suppressed by methylation in most of the non-small-lung cancer cells.

### INDUSTRIAL APPLICABILITY

According to the present invention, a cancer-associated gene to be used as an index for detecting canceration of a cell and degree of malignancy of cancer was found, and a method of detecting cancer using the cancer-associated gene as an index was provided, and furthermore a suppression/therapeutic method of cancer using the cancer-associated gene as essential part was provided.

1. A method for detecting cancer selected from lung squamous cell carcinoma, lung adenocarcinoma or lung large cell carcinoma, wherein canceration of a specimen is detected based on an index of not less than 1.5 fold amplification of at least one gene selected from the group consisting of
MUC1 gene, PRCC gene, EIF4G gene, THPO gene, TERT gene, DAB2 gene, EGFR gene, ELN gene, MUC3 gene, MYC gene, PVT1 gene, FACA gene, PTPN1 gene, Livin gene, MYCN gene, ELK3 gene, BCL2L2 gene, HNF3A gene, ERBB2 gene, and CGI-147 gene;
in comparison with a normal cell.

2. The method according to claim 1, wherein canceration of a specimen is detected based on an index of not less than 4 fold amplification of at least one gene selected from the group consisting of
MYCN gene, MYC gene, PVT1 gene, ELK3 gene, BCL2L2 gene, HNF3A gene, ERBB2 gene, and CGI-147 gene;
in comparison with a normal cell.

3. A method for detecting non-small cell lung cancer, wherein canceration of a specimen is detected based on an index of not less than 1.5 fold amplification of at least one gene selected from the group consisting of
ESP15 gene, MUC1 gene, ARHGEF2 gene, PRCC gene, ABCC5 gene, EIF4G1 gene, CDH12 gene, PC4 gene, SKP2 gene, DAB2 gene, ZNF131 gene, RAD52 gene, WNT3 gene, CDC27 gene, ABCC3 gene, PPM1D gene, ITGB4 gene, BIRC5 gene, SHGC-103396 gene, BCL2L1 gene, RBL1 gene, SRC gene, TGIF2 gene, MYBL2 gene, BIRC7 gene, ARAF1 gene, CUL4B gene, CTAG1 gene, TRIM33 gene, MYCL1 gene, RLF gene, MYC gene, CCNE1 gene, and PCTK1 gene;
in comparison with a normal cell.

4. The method according to claim 3, wherein canceration of a specimen is detected based on an index of not less than 4 fold amplification of at least one gene selected from the group consisting of
TRIM33 gene, MYCL1 gene, RLF gene, MYC gene, CCNE1 gene, and PCTK1 gene;
in comparison with a normal cell.

5. A method for detecting cancer selected from lung squamous cell carcinoma, lung adenocarcinoma or lung large cell carcinoma, wherein canceration of a specimen is detected based on an index of a heterozygous deletion of at least one gene selected from the group consisting of BAIAP1 gene, LZTS1 gene, AAC1 gene, BLK gene, MTAP gene, CDKN2A (p16) gene, GPC5 gene, SNRPN gene, SAMD4 gene, DCC gene, ADRBK2 gene, DBCCR1 gene, and RIZ gene, or an index of a homozygous deletion of at least one gene selected from the group consisting of CDKN2A (p16) gene, MTAP gene, DBCCR1 gene, and RIZ gene.

6. A method for detecting non-small cell lung cancer, wherein canceration of a specimen is detected based on an index of a heterozygous deletion of at least one gene selected from the group consisting of CCK gene, UBE2E1 gene, THRB gene, RARB gene, TGFβR2 gene, MLH1 gene, CTNNB1 gene, VIPR1 gene, ZNF35 gene, TGM4 gene, TDGF1 gene, PTPRG gene, FHIT gene, BAIAP1 gene, MITF gene, LOC151987 gene, EIF4E gene, NFκB gene, CCNA gene, PGRMC2 gene, VEGFC gene, MSH3 gene, RASA1 gene, TPT1 gene, RB1 gene, AATF gene, ADRBK2 gene, and NB2F1 gene, or an index of a homozygous deletion of NR2F1 gene.

7. A method of detecting cancer wherein a non-small cell lung cancer is detected by detecting suppression of DBCCR1 gene expression in a specimen.

8. The detection method according to claim 1, wherein the detection is performed by a CGH method, DNA chip method, quantitative PCR method or real time RT-PCR method.

9. The detection method according to claim 1, wherein the detection is performed by a CGH method or DNA chip method and a plurality of types of DNA fragments to be fixed onto the detection substrate are genomic DNA, cDNA or synthetic oligonucleotides.

10. The detection method according to claim 1, wherein the detection is performed by a CGH method, and a plurality of types of DNA fragments to be fixed onto the detection substrate are genomic DNA, and the genomic DNA is a gene amplification product of BAC DNA, YAC DNA or PAC DNA.

11. The detection method according to claim 3, wherein the detection is performed by a CGH method, DNA chip method, quantitative PCR method or real time RT-PCR method.

12. The detection method according to claim 3, wherein the detection is performed by a CGH method or DNA chip method and a plurality of types of DNA fragments to be fixed onto the detection substrate are genomic DNA, cDNA or synthetic oligonucleotides.

13. The detection method according to claim 3, wherein the detection is performed by a CGH method, and a plurality of types of DNA fragments to be fixed onto the detection substrate are genomic DNA, and the genomic DNA is a gene amplification product of BAC DNA, YAC DNA or PAC DNA.

14. The detection method according to claim 5, wherein the detection is performed by a CGH method, DNA chip method, quantitative PCR method or real time RT-PCR method.

15. The detection method according to claim 5, wherein the detection is performed by a CGH method or DNA chip method and a plurality of types of DNA fragments to be fixed onto the detection substrate are genomic DNA, cDNA or synthetic oligonucleotides.

16. The detection method according to claim 5, wherein the detection is performed by a CGH method, and a plurality of types of DNA fragments to be fixed onto the detection substrate are genomic DNA, and the genomic DNA is a gene amplification product of BAC DNA, YAC DNA or PAC DNA.

17. The detection method according to claim 6, wherein the detection is performed by a CGH method, DNA chip method, quantitative PCR method or real time RT-PCR method.

18. The detection method according to claim 6, wherein the detection is performed by a CGH method or DNA chip method and a plurality of types of DNA fragments to be fixed onto the detection substrate are genomic DNA, cDNA or synthetic oligonucleotides.

19. The detection method according to claim 6, wherein the detection is performed by a CGH method, and a plurality of types of DNA fragments to be fixed onto the detection substrate are genomic DNA, and the genomic DNA is a gene amplification product of BAC DNA, YAC DNA or PAC DNA.

20. The detection method according to claim 7, wherein the detection is performed by a CGH method, DNA chip method, quantitative PCR method or real time RT-PCR method.

21. The detection method according to claim 7, wherein the detection is performed by a CGH method or DNA chip method and a plurality of types of DNA fragments to be fixed onto the detection substrate are genomic DNA, cDNA or synthetic oligonucleotides.

22. The detection method according to claim 7, wherein the detection is performed by a CGH method, and a plurality of types of DNA fragments to be fixed onto the detection substrate are genomic DNA, and the genomic DNA is a gene amplification product of BAC DNA, YAC DNA or PAC DNA.

23. A method for suppressing a non-small cell lung cancer cell, which comprises introducing a gene, whose deletion is involved in canceration of a non-small cell lung cancer cell, into a non-small cell lung cancer cell.

24. A method for suppressing a non-small cell lung cancer cell, which comprises introducing at least one gene selected from a group consisting of MTAP gene, CDKN2A (p16) gene, DBCCR1 gene and 1RIZgene into a non-small cell lung cancer cell.

25. A method for suppressing a small cell lung cancer cell, which comprises introducing a gene, whose deletion is involved in canceration of a small cell lung cancer cell, into a small cell lung cancer cell.

26. A method for suppressing a small cell lung cancer cell, which comprises introducing NR2F1 gene into a small cell lung cancer cell.

27. A method of suppressing a small cell lung cancer cell, which comprises applying, to a small cell lung cancer cell, a nucleic acid antagonizing a transcriptional product of a gene whose amplification is involved in canceration of the small cell lung cancer cell.

28. A method of suppressing a small cell lung cancer, which comprises applying, to a small cell lung cancer, a nucleic acid antagonizing a transcriptional product of at least one gene selected from the group consisting of MYCN gene, MYC gene, PVT1 gene, ELK3 gene, BCL2L2 gene, HNF3A gene, ERBB2 gene and CGI-147 gene

29. The method according to claim 27, wherein the nucleic acid antagonizing a transcriptional product of a gene is small interference RNA against a transcriptional poroduct mRNA, or an antisense oligonucleotide of the mRNA.

30. A method of suppressing a non-small cell lung cancer cell, which comprises applying, to a non-small cell lung cancer cell, a nucleic acid antagonizing a transcriptional product of a gene whose amplification is involved in canceration of the non-small cell lung cancer cell.

31. A method of suppressing a non-small cell lung cancer, which comprises applying, to a non-small cell lung cancer, a nucleic acid antagonizing a transcriptional product of at least one gene selected from the group consisting of TRIM33 gene, MYCL1 gene, RLF gene, MYC gene, CCNE1 gene and PCTK1 gene.

32. The method according to claim 30, wherein the nucleic acid antagonizing a transcriptional product of a gene is small interference RNA against a transcriptional poroduct mRNA, or an antisense oligonucleotide of the mRNA.
